(19) United States
(12) Patent Application Publication
(10) Pub. No.: US 2005/0197365 A1
(43) Pub. Date: Sep. 8, 2005

(54) Diamino thiazoloindan derivatives and their use

(76) Inventors: Jeffrey Sterling, Jerusalem (IL); Liat Hayardeny-Nisimov, Tel Aviv (IL); David Lerner, Jerusalem (IL); Yaacov Herzig, Raanana (IL); Eliezer Falb, Givataim (IL); Gyorgy Toth, Nyiregyhaza (HU); Sandor Molnar, Debrecen (HU); Dalia Pinkert, Kfar Saba (IL)

Correspondence Address: COOPER & DUNHAM, LLP, 1185 AVENUE OF THE AMERICAS, NEW YORK, NY, 10036, US

(21) Appl. No.: 11/067,110

(22) Filed: Feb. 25, 2005

Related U.S. Application Data
(60) Provisional application No. 60/548,223, filed on Feb. 27, 2004.

(51) Int. Cl. A61K 31/428; C07D 277/84

(52) U.S. Cl. 514/366; 548/150

(57) ABSTRACT

The subject invention provides a compound having the structure: 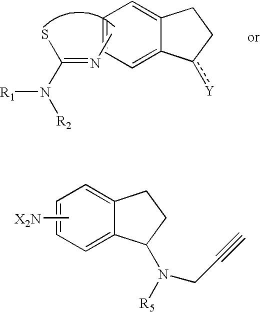
wherein Y is O, NR₃R₄ or NOR₆; R₃ is H, alkyl, aralkyl, alkynyl, trifluoroacetyl, t-butoxycarbonyl or an acyl group; R₄ is H, alkyl, aralkyl, or alkynyl; R₆ is H or C₁-C₄ alkyl; R₁ and R₂ are each independently H, alkyl, aralkyl, or alkynyl; the curved line drawn from S to the center of the phenyl ring and the straight line drawn from N to the center of the ring indicate that S and N are part of a 5 membered ring which shares two carbons with the phenyl ring; and the dashed line drawn from the carbon atom on the cyclopentyl ring to Y represents a bond when Y is O or NOR₆ and is absent when Y is NR₃R₄, and wherein wherein X is H or O; and R₅ is H, alkyl, trifluoroacetyl, t-butoxycarbonyl or an acyl group, or an enantiomer, or a tautomer, or a pharmaceutically acceptable salt thereof, a process for preparing the compounds and a method of treating Parknison's disease, multiple sclerosis or depression with the compounds of the invention.

This application claims the benefit of U.S. Provisional Application No. 60/548,223, filed Feb. 27, 2004, the entire contents of which are hereby incorporated by reference. Throughout this application various publications are referenced in parenthesis. The disclosures of these publications in their entireties are hereby incorporated by reference into this application in order to more fully describe the state of the art to which this invention pertains.

### BACKGROUND OF THE INVENTION

N-Propargyl-(R)-1-aminoindan (rasagiline) mesylate, a highly selective MAO-B inhibitor, is currently being developed as an anti-Parkinsonian drug. In addition to its anti-Parkinsonian activity, this compound has been shown to be neuroprotective in a variety of pharmacological models. It has been reported to enhance cognition, increase survival and prevent stroke in SH rats (S. Eliash et al., J. Neural Transm. (2001) 108: 909-923), enhance SOD and catalase activities in the dopaminergic system in the rat (K. Kitani et al., J. Neural Transm. (2000) Suppl. 60: 139-156), and to regulate amyloid precursor protein (APP)(M. Yogev-Falach et al., Neural Plasticity (2002) 9(2):124). The activity profile of rasagiline may be attributed to the propargylamine pharmacophore attached to the bicyclic indan ring system.

The 2-aminothiazole functionality has been successfully applied as a heterocyclic bioisostere of the phenol moiety in dopamine agonists such as talipexole and pramipexole (Refs. cited in van Vliet, et al., J. Med. Chem., (2000) 43: 3549). The latter compound was reported to have neuroprotective properties related to its antioxidant activity (Hall et al., Brain Res., (1996) 742: 80-88). Some compounds with a 2-aminothiazole moiety have free radical scavenging properties (Bonne et al., Arzneimittel-Forsch. (1989) 39: 1246-1250). Compounds comprising the 2-aminothiazole pharmacophore have been reported as serotonin S₂ antagonists, D₂ dopmaine agonists and as agonists of the human β₃ adrenergic receptor (van Vliet et al., J. Med. Chem. (2000) 43:3459).

2-Aminothiazole derivatives of 2-aminoindans and benzopyrans have been reported as orally active central dopamine partial agonists (van Vliet et al. J. Med. Chem. (2000) 43: 3549).

PCT International Publication No. WO 00/01680 discloses a genus of compounds which includes tricyclic ring systems comprising a heterocycle fused to the aromatic ring of a propargyl-substituted aminoindan, which are asserted to be useful in treating Parkinson's disease and dopamine receptor related central nervous system diseases. However, the compounds disclosed in WO 00/01680 differ from the compounds described below in that the nitrogen atom of the aminoindan moiety is attached at a different ring position. In addition, WO 00/01680 does not describe how to make any compounds having a propargyl-substituted aminoindan moiety and does not disclose that the compounds may be useful for treating multiple sclerosis.

The present invention provides novel diamino dihydrothiaazaindacenes comprising a 2-aminothiazole moiety fused to an indan ring system bearing an amino group in position 1 of the indan five membered ring, their preparation and their use. The inventive compounds have been found to combine the neuroprotective effects of both rasagiline and the 2-aminothiazole pharmacophores.

In addition, the present invention provides novel amino 1-propargylaminoindans and the preparation of these compounds, which can also be used as precursors for the synthesis of the diamino dihydrothiaazaindacenes of the invention.

### SUMMARY OF THE INVENTION

The subject invention provides a compound having the structure: 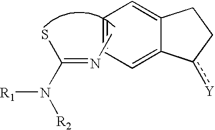
wherein
Y is O, NR₃R₄ or NOR₆;
   R₃ is H, alkyl, aralkyl, alkynyl, trifluoroacetyl, t-butoxycarbonyl or an acyl group;
   R₄ is H, alkyl, aralkyl, or alkynyl;
   R₆ is H or C₁-C₄ alkyl;
R₁ and R₂ are each independently H, alkyl, aralkyl, or alkynyl;
the curved line drawn from S to the center of the phenyl ring and the straight line drawn from N to the center of the ring indicate that S and N are part of a 5 membered ring which shares two carbons with the phenyl ring; and
the dashed line drawn from the carbon atom on the cyclopentyl ring to Y represents a bond when Y is O or NOR₆ and is absent when Y is NR₃R₄,
   or an enantiomer, or a tautomer, or a pharmaceutically acceptable salt thereof.

The subject invention also provides a compound having the structure: 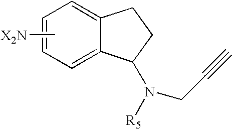
wherein
X is H or O; and
R₅ is H, alkyl, trifluoroacetyl, t-butoxycarbonyl or an acyl group,
   or an enantiomer, or a tautomer, or a pharmaceutically acceptable salt thereof.

The subject invention also provides a compound having the structure: 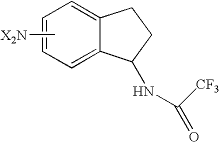
wherein X is H or O.

The subject invention also provides a method of treating a subject suffering from Parkinson's disease or multiple sclerosis, comprising administering to the subject a therapeutically effective amount of a compound of the invention so as to thereby treat the subject.

The subject invention also provides a method for treating a subject suffering from depression comprising administering to the subject a therapeutically effective amount compound I so as to thereby treat the subject.

### DETAILED DESCRIPTION OF THE FIGURES

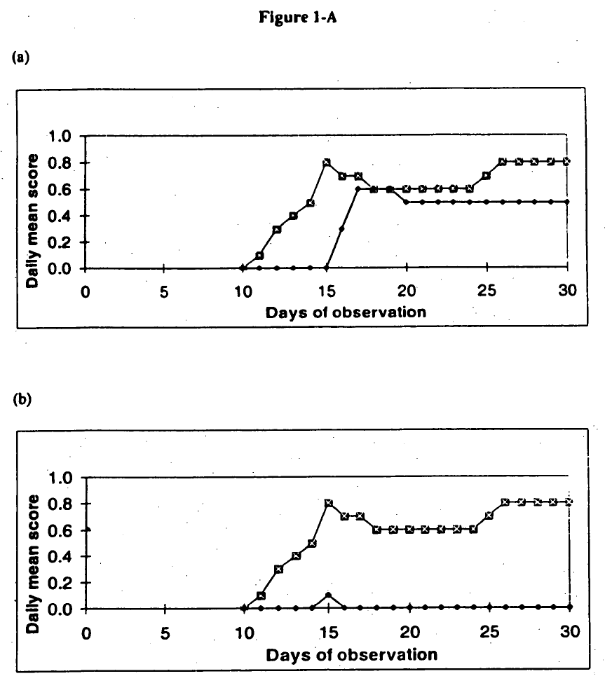

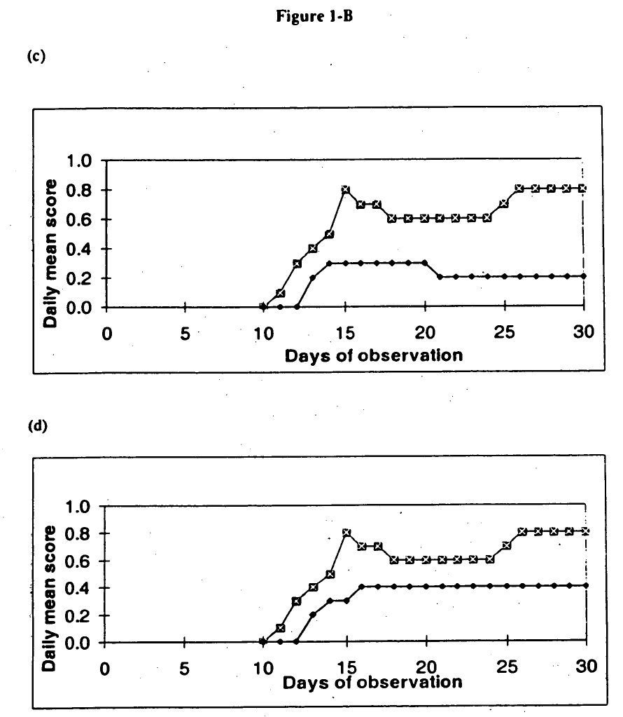

FIGS. 1-A & 1-B show the results of treatment of EAE in mice with Compounds A-1 and B-1 relative to the control group (methyl cellulose) at the following dosages:
(a) A-1 (5 mg/kg twice a day)
(b) B-1 (5 mg/kg twice a day)
(c) A-1 (10 mg/kg twice a day)
(d) B-1 (10 mg/kg twice a day)
-♦- indicates the treated group
-▪- indicates the control group

### DETAILED DESCRIPTION OF THE INVENTION

The subject invention provides a compound having the structure: 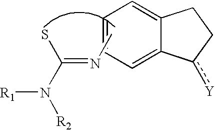
wherein
Y is O, NR₃R₄ or NOR₆;
   R₃ is H, alkyl, aralkyl, alkynyl, trifluoroacetyl, t-butoxycarbonyl or an acyl group;
   R₄ is H, alkyl, aralkyl, or alkynyl;
   R₆ is H or C₁-C₄ alkyl;
R₁ and R₂ are each independently H, alkyl, aralkyl, or alkynyl;
the curved line drawn from S to the center of the phenyl ring and the straight line drawn from N to the center of the ring indicate that S and N are part of a 5 membered ring which shares two carbons with the phenyl ring; and
the dashed line drawn from the carbon atom on the cyclopentyl ring to Y represents a bond when Y is O or NOR₆ and is absent when Y is NR₃R₄,
   or an enantiomer, or a tautomer, or a pharmaceutically acceptable salt thereof.

In one embodiment, the compound has the structure: 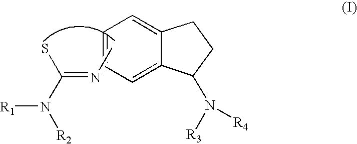
wherein,
R₁, R₂, and R₄ are each independently H, alkyl, aralkyl, or alkynyl;
R₃ is H, alkyl, aralkyl, alkynyl, trifluoroacetyl, t-butoxycarbonyl or an acyl group; and
the curved line drawn from S to the center of the phenyl ring and the straight line drawn from N to the center of the ring indicate that S and N are part of a 5 membered ring which shares two carbons with the phenyl ring,
   or an enantiomer, or a tautomer, or a pharmaceutically acceptable salt thereof.

In a further embodiment,
R₁, R₂, R₃ and R₄ are each independently H, alkyl, aralkyl, or alkynyl.

In yet a further embodiment, R₃ or R₄ is alkynyl.

In a further embodiment, R₃ or R₄ is propargyl.

In another embodiment, the compound has the structure: 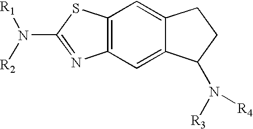
wherein R₁, R₂, R₃ and R₄ are as defined above.

In one embodiment, R₁ is H and R₂ is H.

In another embodiment, R₁, R₂ and R₄ are each H and R₃ is t-butoxycarbonyl.

In another embodiment, R₁ and R₂ are each H, R₃ is t-butoxycarbonyl, and R₄ is alkyl.

In another embodiment, R₁, R₂ and R₄ are each H and R₃ is trifluoroacetyl.

In another embodiment, R₁ and R₂ are each H, R₃ is trifluoroacetyl, and R₄ is alkyl.

In another embodiment, the compound has the structure: 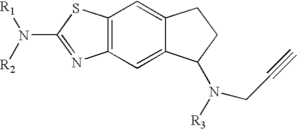
wherein R₁, R₂ and R₃ are as defined above.

In one embodiment, R₁ is H and R₂ is H.

In another embodiment, the compound has the structure: 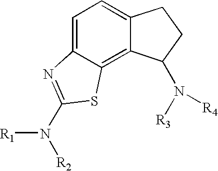
wherein R₁, R₂, R₃ and R₄ are as defined above.

In another embodiment, R₁ is H and R₂ is H.

In one embodiment, the compound has the structure: 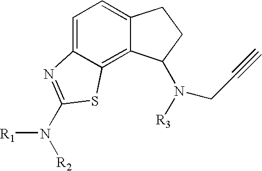
wherein R₁, R₂ and R₃ are as defined above.

In a further embodiment, R₁ is H and R₂ is H.

In a further embodiment of the above compounds R₃ is H.

In another embodiment of the above compounds, R₃ is t-butoxycarbonyl.

In another embodiment of the above compounds, R₃ is trifluoroacetyl.

In another embodiment, R₁, R₂ and R₄ are each H and R₃ is alkyl.

In another embodiment, R₁, R₂, R₃ and R₄ are each H.

In another embodiment, the compound has the structure: 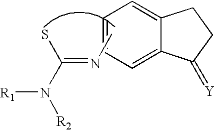
wherein Y is O or NOR₆, and
R₁, R₂ and R₆ are as defined above.

In one embodiment, the compound has the structure:

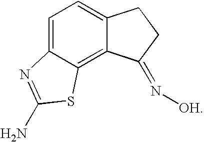

In another embodiment, the compound has the structure:

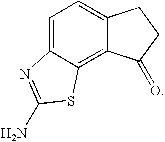

The subject invention also provides a compound having the structure: 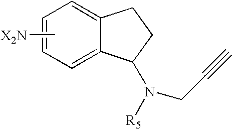
wherein
X is H or O; and
R₅ is H, alkyl, trifluoroacetyl, t-butoxycarbonyl or an acyl group,
   or an enantiomer, or a tautomer, or a pharmaceutically acceptable salt thereof.

In one embodiment,
X is H; and
R₅ is H, alkyl, trifluoroacetyl, or t-butoxycarbonyl.

In another embodiment,
X is O; and
R₅ is H, alkyl, trifluoroacetyl, or t-butoxycarbonyl.

In a further embodiment, the compound has the structure:

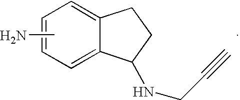

In a further embodiment, the compound has the structure:

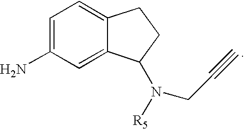

In a further embodiment, the compound has the structure:

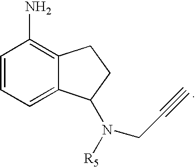

In another embodiment, the compound has the structure:

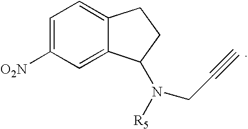

In another embodiment, the compound has the structure:

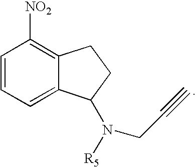

In another embodiment of the above compounds R₅ is H.

In another embodiment of the above compounds R₅ is t-butoxycarbonyl.

In another embodiment, R₅ is trifluoroacetyl.

The subject invention also provides a compound having the structure: 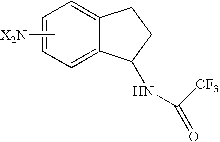
wherein X is H or O.

In one embodiment, X is O.

In another embodiment, X is H.

The subject invention also provides a process for manufacturing a compound having the structure: 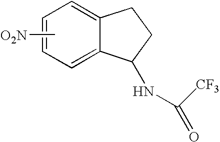
comprising reacting 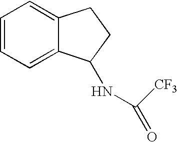
with nitromethane in the presence of sulfuric acid and nitric acid to produce the compound.

The subject invention also provides a process for manufacturing a compound having the structure: 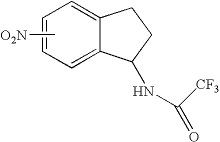
comprising reacting 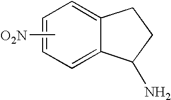
with trifluoroacetic anhydride and potassium hydroxide in the presence of acetonitrile to produce the compound.

The subject invention also provides a process for manufacturing a compound having the structure: 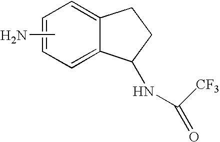
comprising reacting 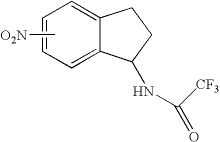
with iron dust and hydrochloric acid in the presence of ethanol to produce the compound.

The subject invention also provides a method of treating a subject suffering from Parkinson's disease or multiple sclerosis, comprising administering to the subject a therapeutically effective amount of compound I so as to thereby treat the subject.

In a further embodiment, the method further comprises administering to the subject a therapeutically effective amount of glatiramer acetate, interferon beta-1b or interferon beta-1a.

In one embodiment of the above method, the subject suffers from Parkinson's disease.

In another embodiment, the subject suffers from multiple sclerosis.

The subject invention also provides a method for treating a subject suffering from depression comprising administering to the subject a therapeutically effective amount compound I so as to thereby treat the subject.

The subject invention also provides a pharmaceutical composition comprising a compound of any one of the above compounds and a pharmaceutically acceptable carrier.

In one embodiment, the pharmaceutical composition further comprises glatiramer acetate, interferon beta-1b or interferon beta-1a.

In one embodiment of the above methods, the therapeutically effective amount of the compound is administered by injection, systemically, orally or nasally.

The subject invention also provides a process for the manufacture of a pharmaceutical composition comprising admixing any of the above compounds with a pharmaceutically acceptable carrier.

The subject invention also provides a packaged pharmaceutical composition for treating Parkinson's disease, multiple sclerosis or depression in a subject, comprising:
(a) a container holding a therapeutically effective amount of any of the above tricyclic compounds with the protecting groups removed; and
(b) instructions for using the compound for treating Parkinson's disease, multiple sclerosis or depression in the subject.

The subject invention also provides a process of manufacturing the compound having the structure: 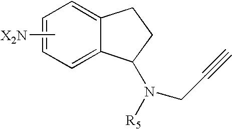
wherein X is H or O and R₅ is H comprising
reacting 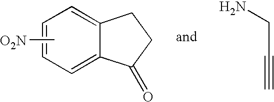
in the presence of Na(OAc)₃BH and DCE to produce

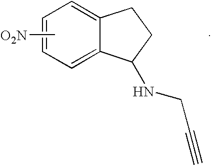

The subject invention also provides a process of manufacturing the above compound, wherein X is H or O and R₅ is H or alkyl, comprising reacting 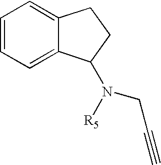
with a nitrating agent in the presence of acid to produce

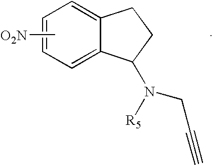

In one embodiment of the above process, R₅ is alkyl.

In another embodiment, R₅ is H.

In another embodiment of the process wherein R₅ is alkyl, the process further comprises reacting the product with a reducing agent in the presence of solvent to produce

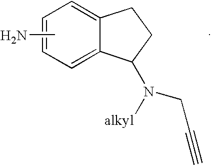

In a further embodiment of the above process, the nitrating agent is HNO₃, CH₃NO₂ or a combination thereof, and the acid is H₂SO₄.

In a further embodiment of the above process, the reducing agent is NaBH₄, SnCl₂, or a combination thereof and the solvent is ethanol.

In one embodiment of the above processes, the process further comprises the steps of
(a) reacting the product with a suitable protecting group in the presence of solvent to produce 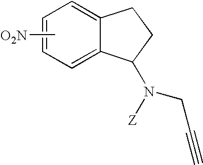
   wherein Z is a protecting group;
(b) reacting the product of step (a) with a reducing agent in the presence of solvent to produce 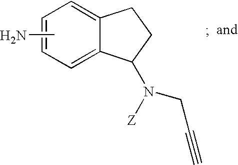
(c) removing the protecting group of the product of step (b) with to produce the compound wherein X is H and R₅ is H.

In a further embodiment, the reducing agent of step (b) is NaBH₄, SnCl₂, or a combination thereof and the solvent is ethanol.

In a further embodiment, the protecting group of step (a) is t-butoxycarbonyl and the solvent is ethanol.

In another embodiment, the protecting group is removed in step (c) by reacting the product of step (b) with HCl in the presence of dioxane.

In another embodiment, the protecting group of step (a) is trifluoroacetyl and the solvent is toluene.

In a further embodiment, the protecting group is removed in step (c) by reacting the product of step (b) with K₂CO₃ in the presence of methanol.

The subject invention also provides a process of manufacturing compound I, comprising the steps of:
(a) reacting a compound having the structure: 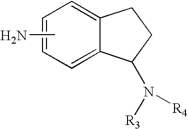
   wherein
R₃ is trifluoroacetyl or t-butoxycarbonyl; and
R₄ is H, alkyl, aralkyl, or alkynyl;
   with cyclizing agents in the presence of solvent; and
(b) removing the trifluoroacetyl or t-butoxycarbonyl group by reacting the product of step (a) with a suitable reagent to produce the compound.

In one embodiment of the above process, R₃ is trifluoroacetyl, R₄ is H or propargyl, and the cyclization agents of step (a) are NH₄SCN and bromine and the solvent is HOAc.

In another embodiment, the trifluoroacetyl group is removed by reacting the product of step (a) with K₂CO₃ in the presence of water and methanol.

In another embodiment, R₃ is t-butoxycarbonyl, R₄ is propargyl, and the cyclization agents of step (a) are NH₄SCN and bromine and the solvent is HOAc.

In another embodiment, the t-butoxycarbonyl group is removed by reacting the product of step (a) with HCl in the presence of dioxane.

The subject invention also provides a process of manufacturing a compound having the structure: 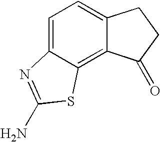
comprising reacting a compound having the structure 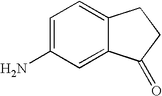
with cyclization agents in the presence of solvent to produce the compound.

The subject invention also provides a process for manufacturing a compound having the structure: 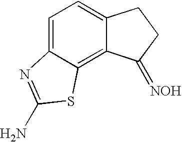
comprising refluxing the compound having the structure: 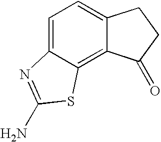
with NH₂OH.HCl, NaOAc, EtOH, and water to produce the compound.

In one embodiment, the cyclization agents used above are NH₄SCN and bromine and the solvent is HOAc.

The subject invention also provides a process of manufacturing the compound having the structure: 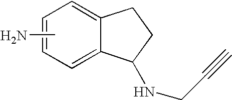
comprising treating the compound having the structure: 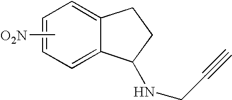
with SnCl₂ in the presence of solvent to produce the compound.

In one embodiment, the solvent is EtOH.

The subject invention also provides a process of manufacturing the above compound comprising treating the compound having the structure: 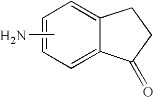
with propargylamine and Na(OAc)₃BH in the presence of solvent to produce the compound.

In one embodiment, the solvent is dichloroethane.

The subject invention also provides a process of manufacturing a compound having the structure: 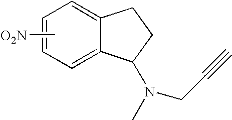
comprising treating a compound having the structure: 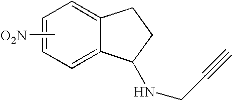
with NaCNBH₃ and paraformaldehyde in the presence of solvent to produce the compound.

In one embodiment, the solvent is MeOH.

The subject invention also provides a process for manufacturing a compound having the structure 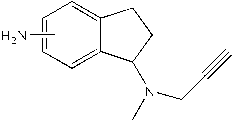
comprising treating the compound having the structure: 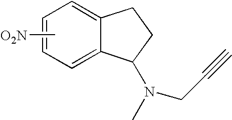
with a reducing agent in the presence of solvent to produce the compound.

In one embodiment, the solvent is EtOH.

In another embodiment, the reducing agent is SnCl₂.

The subject invention further provides a process for manufacturing a compound having the structure: 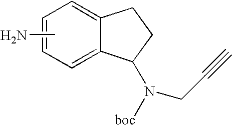
comprising treating the compound having the structure: 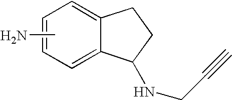
with di-tert-butyl dicarbonate in the presence of solvent.

In one embodiment, the solvent is a mixture of dichloromethane and triethylamine.

The subject invention also provides the use of any of the above tricyclic compounds with the protecting group removed for manufacturing a medicament useful for treating Parkinson's disease or multiple sclerosis in a subject.

In one embodiment, the medicament further comprises a therapeutically effective amount of glatiramer acetate, interferon beta-1b or interferon beta-1a.

In one embodiment, the disease is Parkinson's disease.

In another embodiment, the disease is multiple sclerosis.

The subject invention also provides the use of any of the above tricyclic compounds with the protecting group removed for manufacturing a medicament useful for treating depression in a subject.

The subject invention also provides a pharmaceutical composition for use in treating Parkinson's disease or multiple sclerosis in a subject comprising a therapeutically effective amount of any of the above tricyclic compounds with the protecting group removed and a pharmaceutically acceptable carrier.

In one embodiment, the pharmaceutical composition further comprises a therapeutically effective amount of glatiramer acetate, interferon beta-1b or interferon beta-1a.

The subject invention further comprises a pharmaceutical composition for use in treating depression in a subject comprising a therapeutically effective amount of any of the above tricyclic compounds with the protecting group removed and a pharmaceutically acceptable carrier.

It is noted that compounds A-1 and B-1 can prevent neuronal death and improve the outcome in various models resembling human degenerative disorders.

Zarate et al. (Am. J. Psychiatry (2004) 161: 171-174) have recently shown that riluzole has antidepressant properties in some patients. The compounds of the invention have the active portions of riluzole. Consequently, the compounds of the present invention are reasonably expected to have antidepressant properties.

It is further noted that the compounds of the invention may be used in addition to levodopa therapy for Parkinson's disease or in addition to glatiramer acetate (the drug substance of Copaxone) or interferon beta-1b or interferon beta-1a, e.g. for multiple sclerosis.

Those skilled in the art will be familiar with the fact that some compounds of the formula (I) can exist as tautomers. The compounds of the formula (I) are therefore also to be understood as meaning herein the relevant tautomers, even when not mentioned specifically in each individual case. This invention also relates to the use of all such tautomers and mixtures thereof.

It will be noted that the structure of some of the compounds of this invention includes asymmetric carbon atoms and thus occur as racemic mixtures and single enantiomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis.

As set out above, certain embodiments of the present compounds can contain a basic functional group, such as amino or alkylamino, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

When the compounds of the present invention are administered as pharmaceuticals, to humans and mammals, they can be given per se or as a pharmaceutical composition containing, for example, 0.1 to 99.5% (or 0.5 to 90%) of active ingredient in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, or from about 5 per cent to about 70 per cent, or from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal,intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The invention is further illustrated by the following examples which in no way should be construed as being further limiting. The contents of all references, pending patent applications and published patent applications, cited throughout this application, including those referenced in the background section, are hereby incorporated by reference. It should be understood that the models used throughout the examples are accepted models and that the demonstration of efficacy in these models is predictive of efficacy in humans.

This invention will be better understood from the Experimental Details which follow. However, one skilled in the art will readily appreciate that the specific methods and results discussed are merely illustrative of the invention as described more fully in the claims which follow thereafter.

Experimental Details

Synthesis of Compounds

A general synthesis scheme for synthesis of the compounds of the invention is presented in Scheme I.

Dihydro-thia-aza-diamino-indacenes were obtained by reacting suitably protected amino-1-propargylaminoindan derivatives (eg 5(s6) or 7(s6), Scheme II)) with ammonium thiocyanate and bromine, followed by removal of the protecting group (see Scheme III). Thus, A-1 and B-1 were obtained from either 5(s6) or 7(s6). The protected 6-amino-1-propargylaminoindans 5(s6) and 7(s6) and the corresponding 4-isomers 5(s4) and 7(s4) were obtained as follows (Scheme II): The starting nitro propargylaminoindans were obtained either by reductive propargylamination of nitro indanones or by first reductive amination to the primary amines followed by propargyaltion with propargyl bromide, or by regioselective nitration of propargylaminoindans. The benzylic amine nitrogen was then protected by a suitable protecting group (eg t-butoxycarbonyl, trifluoroacetyl, etc). Finally, reduction of the nitro moiety afforded compounds 5(s6,s4) and 7(s6,s4). This reduction is effected by suitable reducing agents, eg tin chloride or a combination of tin chloride and sodium borohydride (in ethanol as the reaction medium), so as to selectively reduce the nitro group while leaving the triple bond intact.

Compounds 8(s6,s4) may be readily obtained by removing the protecting groups by using methods known to those skilled in the art. Compounds 8(s6,s4) may also be prepared by reductive propargylamination of aminoindanones 13(s6,s4), eg by reacting the latter with propargylamine under reducing conditions such as Na(OAc)₃BH in a non protic solvent such as dichloroethane.

Compounds 8 were also prepared by direct reduction (eg tin chloride, or tin chloride with sodium borohydride) of the nitro precursors 3. N1-alkyl analogues of 8 (25) were obtained by analogous reduction of the corresponding nitro derivatives 24. The latter were prepared by reductive alkylation (eg sodium cyanoborohydride and paraformaldehyde) of 4- and 6-nitro propargylaminoindans 3.

Alternatively, dihydro-thia-aza-diamino-indacenes were prepared by reacting suitably protected amino-1-aminoindan derivatives, e.g. 12(s6), with ammonium thiocyanate and bromine, followed by removal of the protecting group (see Scheme III) and propargylation by reacting e.g. with propargyl halides.

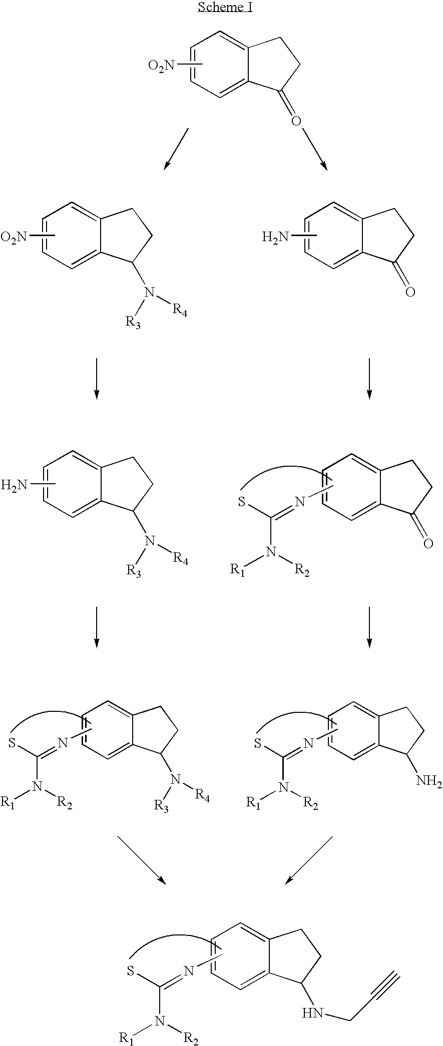

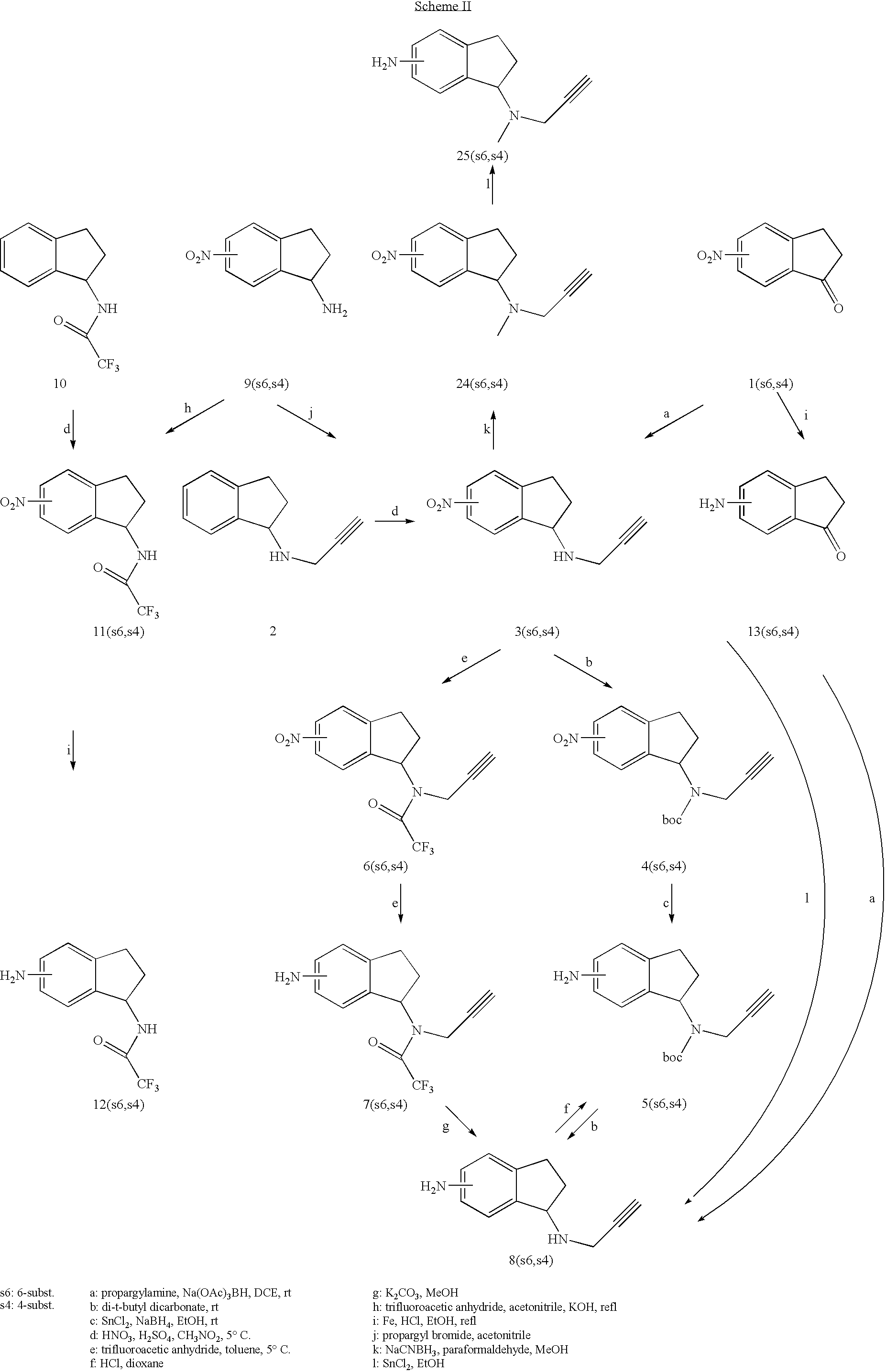

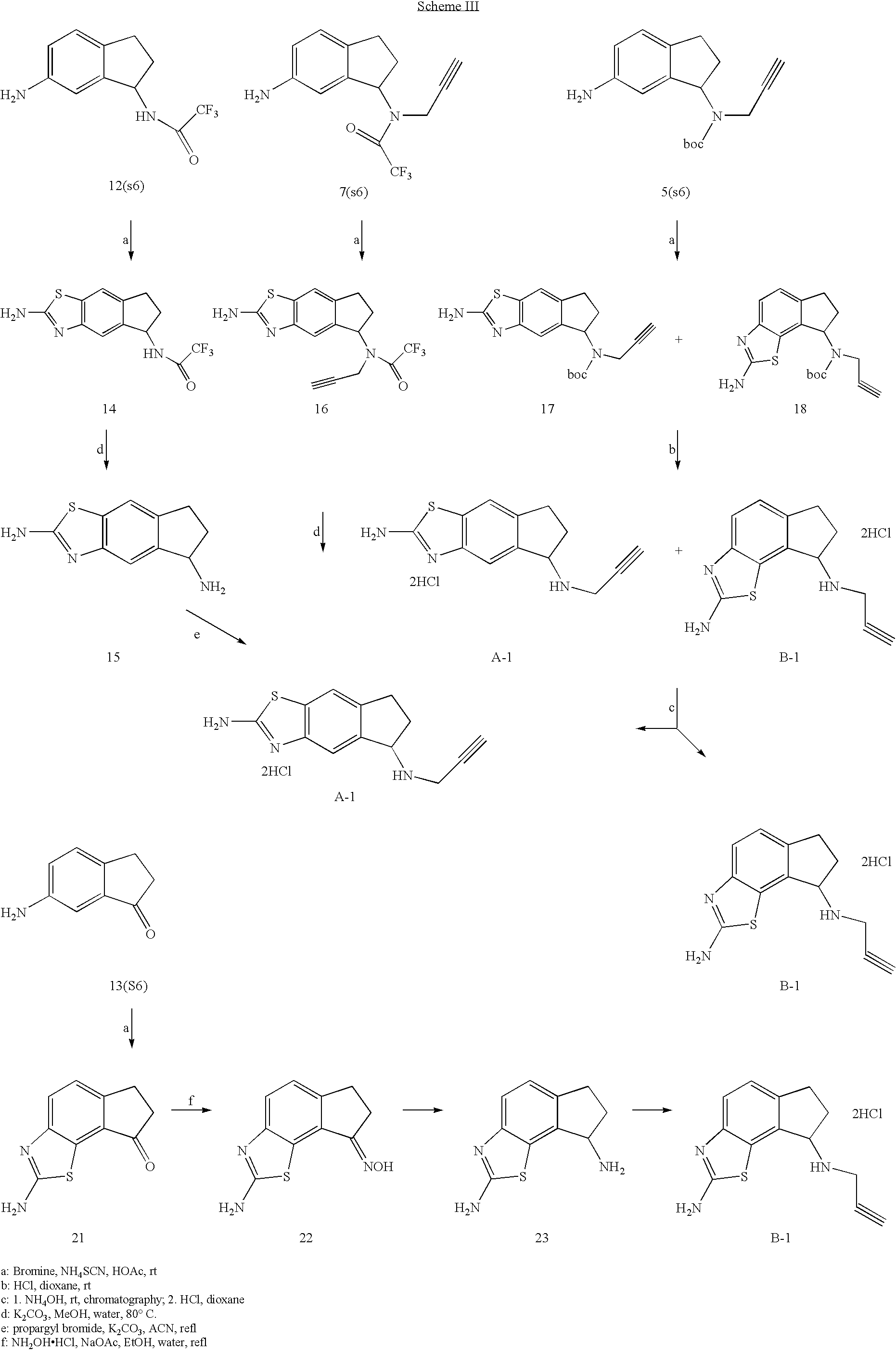

Specific compounds can be prepared as described in the following examples.

### EXAMPLE 1

### (6-Nitro-indan-1-yl)-prop-2-ynylamine (3(s6))

6-Nitroindanone (6.86 g, 38.72 mmol) was dissolved in 1,2 dichlorethane (220 mL), and a solution of propargylamine (2.68 g, 48.66 mmol) in dichloroethane (15 mL) was added. The mixture was stirred at 25° C. under nitrogen for 30 min and sodium triacetoxyborohydride (13.42 g, 63.32 mmol) was added neat. The mixture was then stirred at 25° C. under nitrogen for 50 h. Solvent was evaporated under reduced pressure to give a dark solid residue. The residue was treated with ethyl acetate (300 mL) and the mixture was stirred at 45° C. for 1 h and filtered. Silica gel was added to the filtrate and the mixture was evaporated to dryness under vacuum to give silica gel impregnated with the crude product. This was placed on top of a silica gel column and purified by flash column chromatography (hexane:ethyl acetate 25:75) to give 5.80 g (69%) of a brown solid, mp 37-39° C.

¹H NMR δ (CDCl₃) : 8.20 (s, 1H), 8.10 (dd, 1H), 7.35 (d, 1H), 4.50 (m, 1H, C1-H), 3.55, (m, 2H, CH₂C≡CH), 3.1 (m, 1H, C3-H), 2.9 (m, 1H, C3-H′), 2.5 (m, 1H, C2-H), 1.9 (m, 1H, C2-H′), 2.30 (s, 1H, CH₂C≡CH) ppm.

### EXAMPLE 2

### N-Boc-(6-nitro-indan-1-yl)-prop-2-ynylamine (4(s6))

(6-Nitro-indan-1-yl)-prop-2-ynylamine (6.0 g, 27.74 mmol) was dissolved in absolute ethanol (130 mL) and a solution of di-t-butyl dicarbonate (6.24 g, 28.56 mmol) in absolute ethanol (30 mL) was added dropwise with stirring over 15 min. The solution was then stirred at 25° C. under nitrogen for 24 h. The solvent was evaporated to dryness under reduced pressure to give a dark viscous oil. Hexane (70 mL) was added to the viscous oil and the mixture was stirred for 20 min and the hexane was decanted off. This procedure (adding hexane, stirring for 20 min and decanting off the hexane) was repeated nine more times. The combined hexane washings were evaporated to dryness under reduced pressure to give 8.20 g (93%) of a light tan solid, mp 56-59° C.

¹H NMR δ (CDCl₃) : 8.10 (m, 2H), 7.27 (d, 1H), 5.75,5.18 (m, 1H, C1-H), 4.3-3.5 (m, 2H, CH₂C≡CH), 3.12 (m, 1H, C3-H), 2.92 (m, 1H, C3-H′), 2.55 (m, 1H, C2-H), 2.30 (m, 1H, C2-H′), 2.15 (s, 1H, CH₂C=-CH) ppm.

### EXAMPLE 3

### N-Boc-(6-amino-indan-1-yl)-prop-2-ynylamine (5(s6))

N-Boc-(6-nitro-indan-1-yl)-prop-2-ynylamine (5.55 g, 17.54 mmol) and stannous chloride dihydrate (19.76 g, 87.59 mmol) were dissolved in anhydrous ethanol (320 mL) and heated to 60° C. Sodium borohydride (1.33 g, 35.16 mmol) dissolved in ethanol (70 mL) was then added dropwise with stirring under nitrogen over 30 min. The stirred mixture was heated at 60° C. for 1.5 h, cooled to 10° C., diluted with cold water and the pH was adjusted to 7-8 by 25% NH₄0H, and EtOAc was added. The mixture was stirred for 10 min, filtered, water and brine were added, and the layers were separated; the aqueous layer was re-extracted with EtOAc. The combined organic layers were dried and evaporated to dryness under reduced pressure to give a crude viscous oil which was purified by flash column chromatography (hexane:ethyl acetate 50:50), to give 3.8 g (75 %) of a viscous yellow oil.

¹H NMR δ (CDCl₃) : 7.0 (d, 1H), 6.55 (d, 1H), 6.45 (s, 1H), 5.66, 5.35 (m, 1H, C1-H), 4.2-3.3 (m, 4H, CH₂C≡CH, NH₂), 2.90 (m, 1H, C3-H), 2.70 (m, 1H, C3-H′), 2.40 (m, 1H, C2-H), 2.18 (m, 1H, C2-H′), 2.10 (s, 1H, CH₂C≡CH) ppm.

MS (FAB): 287 (MH⁺, 75), 229 (100), 183 (24), 171 (22). Elemental analysis: calc. C, 71.30; H, 7.75; N, 9.78. Found: C, 71.23; H, 7.59; N, 9.64

### EXAMPLE 3a

### N-Boc-(6-amino-indan-1-yl)-prop-2-ynylamine (5(s6))

6-Amino-1-N-propargylaminoindan dihydrochloride (0.66 g, 2.5 mmol) was dissolved in a mixture of dichloromethane (15 ml ) and triethylamine (1 ml). The solution was cooled at 0-5° C. and di-tert-butyl dicarbonate (Boc₂O) (0.65 g, 2.5 mmol) was added dropwise. The clear solution was stirred for 24 hr at rt, evaporated to dryness, and the yellow crude product obtained was chromatographed on silica gel (20% ethyl acetate in hexane) to afford the title compound as a colorless oil (0.53 g, 74%), identical to that obtained in Example 3.

### Example 4

### N-Trifluoroacetyl-(6-amino-indan-1(S)-yl)-prop-2-ynylamine (S-7(s6))

### 4.1 (6-Nitro-indan-1-yl)-prop-2-ynylamine (S-3(s6))

Sulfuric acid (124 mL) was added dropwise to diluted nitric acid (prepared by adding 65% nitric acid (9.1 mL) to water (19.4 ml)), under cooling and stirring. This nitration mixture was cooled to 2-8° C. and added dropwise to a solution of 1-(S)-propargylaminoindan (17.1 g, 0.1 mol) in nitromethane (180 ml) at 2-8° C. The reaction mixture was stirred at this temperature for 1.5 h, then poured onto a mixture of ice and water (1.8 kg). The mixture was adjusted to pH 8-9 with 40% NaOH and extracted with dichloromethane (2×200 mL). The combined organic phase was dried, filtered, and evaporated to dryness under reduced pressure. Isopropanol (50 mL) was added to the residue and the solution was evaporated to dryness, to give the product as an oil (20.2 g, 93.4%). TLC: R_{f}:0.42 (toluene/EtOAc 3:1)

### 4.2 N-Trifluoroacetyl-(6-nitro-indan-1-(S)-yl)-prop-2-ynylamine (S-6(s6))

A mixture of toluene (14 mL) and compound S-3(s6) (36.8 g, 0.17 mol) added to a mixture of toluene (136 mL) and trifluoroacetic anhydride (39.9 g, 0.19 mol) dropwise at 0-5° C. The reaction mixture was stirred at this temperature for 3.5 h. Potassium hydroxide (13.5 g; 0.24 mol) in water 108 (mL) was added gradually to the reaction mixture under cooling. The mixture was stirred at rt for 2 h and the phases were separated. The aqueous phase was extracted with toluene (2×200 mL). The combined organic phase was evaporated to dryness in vacuo. Isopropanol (50 mL) was added to the residue and the solution was evaporated to dryness. The crude product was crystallized from MeOH to give 26.3 g (49.5%), mp: 83-85° C. TLC=R_{f}: 0.72 (toluene/EtOAc 3:1).

### 4.3 N-Trifluoroacetyl-(6-amino-indan-1-(S)-yl)-prop-2-ynylamine (S-7(s6))

A mixture of compound S-6(s6) (31.2 g, 0.1 mol), SnCl₂.2H₂O (112.8 g, 0.5 mol) and EtOH (1000 mL) was stirred and heated to 600. Sodium borohydride (7.6 g, 0.2 mol) in EtOH (300 mL) was then added over a period of 2 h at 60° C. The reaction mixture was stirred at 60° C. for half an hour then cooled to 5° C. Cold water (1000 mL) was added, and the mixture was adjusted to pH 7-8 with 25% ammonium hydroxide, and extracted with CH₂Cl₂ (2×200 mL). The combined organic phase was dried and evaporated to dryness under reduced pressure to give the title compound (26.4 g; 93.5%) as an oil.

¹H NMR δ (DMSO-d₆) : 6.95 (d, 1H), 6.55 (d, 1H), 6.40, 6.35 (s, 1H), 5.75, 5.38 (m, 1H, C1-H), 4.25, 4.10, 3.75, 3.30 (2H, CH₂C≡CH), 3.3, 3.18 (s, 1H, ≡CH), 2.90 (m, 1H, C3-H), 2.70 (m, 1H, C3-H′), 2.40 (m, 1H, C2-H), 2.25 ppm.

### EXAMPLE 5

### (6-Amino-indan-1-yl)-prop-2-ynylamine (8(B6)) 5.1 From 5(s6)

Compound 8(s6) may be prepared by removing the protecting groups from compounds 5(s6) and 7(s6), by using methods known to those skilled in the art, e.g. subjecting them to acid hydrolysis (HCl in dioxane, trifluoroacetic acid, etc).

### 5.2 From 13(s6)

Sodiumtriacetoxyborohydride (3.70 g, 0.017 mol) was added to a stirred solution of 6-aminoindanone (1.47 g, 0.01 mol) and propargylamine (0.87 ml, 0.013 mol) in dichloroethane (30 ml). The resulting suspension was stirred at rt for 24 h, and additional portions of propargylamine (0.43 ml, 0.0065 mol) and sodiumtriacetoxyborohydride (1.8 g, 0.0035 mol) were added, and stirring continued for 24 h. The reaction mixture was diluted with dichloroethane, washed with 1N NaOH and water, dried and evaporated. The residue was purified by chromatography (silica, EtOAc) to give 1.39 g (75%) of the free base. The latter was converted to the HCl salt (HCl/Et₂O). The crude salt was crystallized from MeOH/iPrOH to give 1.13 g(63%), mp: 216-8° C.

¹H-NMR (CDCl₃) δ : 7.02 (d, 1H), 6.71 (d, 1H), 6.57 (dd, 1H), 4.32 (t, 1H, C1-H), 3.51 (dAB, 2H, CH₂C≡CH), 2.9 (m, 1H, C3-H), 2.72 (m, 1H, C3-H′), 2.72 (br s, 2H), 2.27 (m, 1H, C2-H), 2.26 (t, 1H, CH₂C≡CH), 1.82 (m, 1H, C2-H′) ppm.

### 5.3 From 7(s6)

Compound 8(s6) may be obtained from 7(s6) by removing the trifluoroacetyl protecting group, eg by using potassium carbonate in aqueous MeOH.

### EXAMPLE 6

### (4-Nitro-indan-1-yl)-prop-2-ynylamine HCl (3(s4))

To 4-nitroindanone (10.6 g, 0.06 mol) and propargylamine (4.15 g, 0.075 mol) in 1,2-dichloroethane (330 ml), was added sodium triacetoxyborohydride (20.7 g, 0.098 mol) under nitrogen at rt. The mixture was stirred at rt for 50 hr, and the product was extracted with 0.5 N HCl (350 ml). The aqueous layer was separated, cooled to 0-5° C., and the solid collected by filtration and dried (9.2 g, 60.7%), mp 220-222° C. A second crop (4.8 g, 31.7%) was obtained by evaporation of the mother liqueur at 70-80% and collecting the white solid which precipitated upon cooling. Total yield: 14.0 g (92.4%).

¹H NMR δ (DMSO-d₆) : 10.25 (br s, 2H, NH₂+), 8.21 (d, 1H, Ar), 8.19 (d, 1H, Ar), 7.62 (t, 1H, Ar), 4.94 (m, 1H, C1-H), 3.96 (m, 2H, CH₂C≡CH), 3.74 (m, 1H, ≡CH), 3.58 (m, 1H, C3-H), 3.30 (m, 1H, C3-H′), 2.45 (m, 1H, C2-H), 2.35 (m, 1H, C2-H′) ppm.

### EXAMPLE 7

### N-Boc-(4-nitro-indan-1-yl)-prop-2-ynylamine (4(s4))

(4-Nitro-indan-1-yl)-prop-2-ynylamine HCl (5.05 g, 0.02 mol) and triethylamine (4.0 g, 0.04 mol) were dissolved in 25 ml methanol and cooled at 10-15° C. Di-tert-butyl-dicarbonate (Boc₂O, 4.8 g, 0.022 mol) in 5 ml methanol was added dropwise and the solution was stirred at rt for 24 hr. The reaction mixture was evaporated to dryness, and the residue was washed with hexane and dried, to give 5.0 g (79.3%) of the title compound.

### EXAMPLE 8

### N-Boc-(4-amino-indan-1-yl)-prop-2-ynylamine (5(s4))

### 8.1 Reduction with Tin Chloride and NaBH₄

Compound 4(s4) (5.0 g, 15.8 mmol) and tin chloride (15.0 g, 79 mmol, 5 eq.) were dissolved in 100 ml ethanol at 60-65° C., and a solution of NaBH₄ (1.2 g, 31.6 mmol, 2 eq.) in 50 ml ethanol was added dropwise over 1 hr. The suspension was stirred for 0.5 hr and cooled to rt. The pH was adjusted to 9-10 by addition of triethylamine, the inorganic salt was filtered off, and the filtrate evaporated to dryness. The product was extracted with ethyl acetate (2×150 ml), filtered and the filtrate washed with water. The organic layer was separated, filtered, dried over sodium sulfate and evaporated to dryness to afford 2.2 g, (48.9%) of the title product.

¹H NMR δ (DMSO-d₆), two rotamers : 6.89 (t, 1H, Ar), 6.46 (d, 1H, Ar), 6.31 (br s, 1H, Ar), 5.55, 5.28 (br s, 1H, C1-H), 4.95 (br s, 2H, NH₂), 4.02, 3.81,3.54,3.35 (m, 2H, CH₂C≡CH), 3.05 (s, 1H, ≡CH), 2.76 (m, 1H, C3-H), 2.50 (m, 1H, C3-H′), 2.30 (br s, 1H, C2-H), 2.05 (br s, 1H, C2-H′), 1.46,1.32 (s,s, 9H, tBu) ppm.

### 8.2 Reduction with Tin Chloride

A solution of compound 4(s4) (8.5 g, 0.027 mol) and tin chloride dihydrate (30.3 g, 0.134 mol, 5 eq.) in 200 ml ethanol was stirred overnight at rt. Work-up as in 8.1 afforded 5.5 g (71.2%), of which 4.2 g (54.4%) of white solid was obtained by column chromatography. Mp: 123.7-124.5° C., NMR as in 8.1

### EXAMPLE 9

Starting from (4-nitro-indan-1-yl)-prop-2-ynylamine, compounds 6(s4), 7(s4) and 8(s4) may be prepared according to the procedures described in Examples 4-5.

### EXAMPLE 10

### N₅-Boc,N₅-prop-2-ynyl-6,7-dihydro-5H-1-thia-3-aza-s-indacene-2,5-diamine (17) and N₈-Boc,N₈-prop-2-ynyl-7,8-dihydro-6H-1-thia-3-aza-as-indacene-2,8-diamine (18)

N-Boc-(6-amino-indan-1-yl)-prop-2-ynylamine (5(s6), 10.5 g, 36.67 mmol) was dissolved in glacial acetic acid (80 ml), and ammonium thiocyanate (6.53 g, 85.8 mmol) was added in one portion. The reaction mixture was stirred at rt until a clear solution was obtained, and a solution of bromine (6.53 g, 40.86 mmol) in glacial acetic acid (35 ml) was added dropwise over 45 min under a nitrogen atmosphere, while maintaining the temperature at 25-30° C. Stirring was continued for 3.5 h at rt, and water (600 ml) was added, followed by careful addition of sodium carbonate to pH 10. The resultant mixture was stirred at rt for 5 h, and the precipitated solid was collected by filtration, washed with water and hexane and dried to give 12.5 g of a tan solid. This crude product was subjected to Boc-deprotection (Ex. 9) without further purification. A sample (2 g) of the crude product was purified by column chromatography (silica, EtOAc:hexane 70:30) to give 1.77 g of a 3:1 mixture of 17 and 18, as a white solid.

¹H-NMR (DMSO-d₆) δ : 7.5 (s, 1H, Ar 17), 7.35 (s, 3H, Ar and NH₂ 17), 7.22 (d, 1H, Ar 18), 7.08 (d, 1H, Ar 18), 5.2-5.8 (three br s, 1H, C1-H), 3.25 (s, 2H, NHCH₂), 3.15 (t, 1H, CH₂C≡CH), 3.0 (m, 1H, C3-H), 2.75 (m, 1H, C3-H′), 2.40 (m, 1H, C2-H), 2.18 (m, 1H, C2-H′), 1.05, 1.03 (9H, t-Bu) ppm.

### EXAMPLE 11

### N₅-prop-2-ynyl-6,7-dihydro-5H-1-thia-3-aza-s-indacene-2,5-diamine diHCl (A-1) And N₈-prop-2-ynyl-7,8-dihydro-6H-1-thia-3-aza-as-indacene-2,8-diamine diHCl (B-1)

The crude Boc-protected compound mixture obtained in Ex. 10 (12.5 g, 36.3 mmol) was dissolved in dioxane (450 ml), and a 20% w/w solution of HCl gas in dioxane (250 ml) was added. The reaction mixture was stirred under nitrogen atmosphere at rt for 10 h, and evaporated to dryness to give a yellow solid which was dissolved in a mixture of water (600 ml) and dichloromethane (600 ml). The mixture was filtered, and the layers separated. The aqueous phase was washed with dichloromethane (3×100 ml) and evaporated to dryness to give a yellowish solid. The latter was treated with warm hexane (150 ml), then with warm Et₂O (150 ml), to give after drying 11.5 g (98%) of the title compounds (mixture) as a white solid.

Separation and Purification of the Two Regioisomers:

The above salt (11.5 g, 36.3 mmol) was converted to the free base as follows: A solution of the salt in water (400 ml) and ammonia solution (300 ml), was extracted by CH₂Cl₂. The organic phase was separated, and the aqueous phase was thoroughly extracted with CH₂Cl₂ . The organic extracts were combined, dried and evaporated to dryness to give 8.8 g (quantitative) of a viscous oil. The latter was flash-chromatographed (95/5 CH₂Cl₂/MeOH) to give N₅-prop-2-ynyl-6,7-dihydro-5H-1-thia-3-aza-s-indacene-2,5-diamine (A-1 free base, 5.45 g) and N₈-prop-2-ynyl-7,8-dihydro-6H-1-thia-3-aza-as-indacene-2,8-diamine (B-1 free base, 1.4 g). The two target di-HCl salts were obtained by acidifying (20% HCl in dioxane) of dioxane solutions of the two free bases. The crude salts were purified by dissolving them in water, washing the aqueous solutions with CH₂Cl₂, and finally evaporating to dryness. A-1 (7.3 g) was crystallized from MeOH/Et₂O to give 6.2 g (87.5%) of a white solid, mp: 223-224° C. B-1 was obtained as an off-white solid (1.6 g, (88%), mp: 242-3 ° C.

¹H-NMR (DMSO-d₆δ) of A-1: 10.20 and 9.82 (two br d, 3H, NH₂⁺ and NH⁺), 7.89 (s, 1 H, Ar), 7.82 (s, 1H, Ar), 4.85 (br s, 1H, C1-H), 3.93 (br s, 2H, NH—CH₂), 3.75 (t, 1H, CH₂C≡CH), 3.20 (m, 1H, C3-H), 2.87 (m, 1H, C3-H′), 2.44 (m,1H, C2-H), 2.30 (m, 1H, C2-H′) ppm. MS (CI) (NH₃) m/z (244, MH⁺).

¹H-NMR (DMSO-d₆δ) of B-1: 10.20 and 9.50 (two br d, 3H, NH₂⁺ and NH⁺), 7.53 (d, 1H, Ar), 7.40 (d, 1H, Ar), 4.92 (br d, C1-H), 3.90 (m, 2H, NH—CH₂), 3.80 (m, 2H, CH₂C≡CH and C3-H), 3.40 (m, 1H, C3-H′), 2.88 (m, 1H, C2-H), 2.42 (m, 1H, C2-H′), ppm. MS (CI) (NH₃) m/z (244, MH⁺)

### EXAMPLE 12

### N₅-prop-2-ynyl-6,7-dihydro-5H-1-thia-3-aza-s-indacene-2,5-diamine 2HCl (A-1)

### 12.1 N₅-trifluoroacetyl,N₅-prop-2-ynyl-6, 7-dihydro-5H-1-thia-3-aza-s-indacene-2,5-diamine (16)

6-Amino-N-trifluoroacetyl, N-propargylaminoindan (7(s6), 2.27 g, 8.04 mmol) and NH₄SCN (1.44 g, 18.9 mmol) were dissolved in AcOH (30 ml) and then Br₂ (1.44 g in 9 ml AcOH, 9 mmol) was added within 1 h at rt, and stirred for 24 h. Water (200 ml) was added, and the acidic mixture was neutralized with Na₂CO₃ to pH 10. The crude product precipitated as a sticky gum which solidified on treatment with EtOH and MeOH (total of 100 ml) at 80° C. for 1 h. After cooling to rt, the product was collected by filtration, washed well with water and dried to give a brown solid (2.34 g, 86%).

1H NMR (mixture of isomers and rotamers, DMSOd₆) δ: 7.59 (br d, 1H, Ar major isomer), 7.46 (br d, 2H, NH₂ major isomer), 7.31 (br t, 1H, J=8 Hz, Ar minor isomer), 7.18 (br t, 1H, J=8 Hz, Ar minor isomer), 7.12 (br s, 1H, Ar major isomer), 5.95 and 5.67 (two t, 1H, J=8 Hz, CHN of minor isomer), 5.75 and 5.57 (two t, 1H, J=8 Hz, CHN of major isomer), 4.3-3.5 (four br ABq, 2H, NCH₂ for both isomers and rotomers), 3.4 (m, 4H, CHCH₂CH₂ for both isomers), 3.03 (m, 1H, CHCH₂CH₂ for both isomers), 2.91 (m, 1H, CHCH₂CH₂ for both isomers), 2.45 (m, 1H, CHCH₂CH₂ for both isomers), 2.30 (m, 1H, CHCH₂CH₂ for both isomers).

¹³C (DMSOd₆ for both isomers) δ: 166.43, 166.27, 165.91 (NCNH₂), 156.23, 155.97, 155.75, 155.28 (Ar), 152.16, 151.98 (Ar), 136.85, 136.76, 136.11, 136.04 (Ar), 132.10, 131.64, 130.61 (Ar), 125.81, 122.24, 122.03 (Ar), 118.42, 117.17, 116.88, 112.88 (Ar CH), 79.27, 78.72, 75.01, 74.18, 73.83 (NCH₂CCH), 63.11, 62.67, 62.20 (CHNH₂), 34.68, 32.85, 32.39 (NCH₂), 30.37, 30.11, 29.71, 29.44, 29.10, 28.78 (CHCH₂CH₂). MS (CI) (NH₃) m/z (338, M-H)⁺.

### 12.2 N₅-prop-2-ynyl-6,7-dihydro-5H-1-thia-3-aza-s-indacene-2,5-diamine 2HCl (A-1)

N₅-trifluoroacetyl,N₅-prop-2-ynyl-6, 7-dihydro-5H-1-thia-3-aza-s-indacene-2,5-diamine (16, 2.2 g, 6.5 mmol) and K₂CO₃ (1.2 g, 8.8 mol) were suspended in a MeOH/water mixture (16:11 ml), and the suspension was stirred at 80° C. for 4.5 h. The mixture was evaporated, and the reside was partitioned between EtOAc (100 ml) and water (40 ml). The organic phase was dried and evaporated to leave a brown oil (mixture of two regioisomers). Flash chromatography (CH₂Cl₂:MeOH 9:1) afforded the free base of the title compound (major component) as a brown oil (0.66 g, 42%), and 0.26 g (16.5%) of the minor regioisomer. The major isomer was dissolved in a 2:1 isopropanol:EtOH (15 ml) mixture, and HCl/EtOH (1.5 ml, 28%) was added. Immediate precipitation occurred and Et₂O (20 ml) was added to complete precipitation. The suspension was stirred at rt for 2 h, filtered, and the collected solid was washed with Et₂O and dried to give 825 mg (40%).

¹H NMR (DMSOd₆) δ: 10.33 and 10.05 ( two br s, 4H, NCNH₂⁺ and NH₂⁺), 7.91 and 7.83 (two s, 2H, Ar), 4.88 (br s, 1H, CHN), 3.94 (br ABq, NCH₂), 3.78 (t, 1H, J=2 Hz, CH₂CCH), 3.23 (br dt, 1H, J=16, 7 Hz, CHCH₂CH₂), 2.91 (br ddd, 1H, J=16, 8, 4 Hz, CHCH₂CH₂), 2.52 (br dt, 1H, J=16, 7 Hz, CHCH₂CH₂), 2.38 (m, 1H, CHCH₂CH₂).

¹³C (DMSOd₆) δ: 168.88 (NCNH₂), 140.91, 137.81, 136.81, 125.96 (Ar), 119.24, 111.80 (Ar CH), 79.73, 75.07 (NCH₂CCH), 60.30 (CHNH₂), 33.71 (NCH₂), 29.61 (CHCH₂CH₂), 28.40 (CHCH₂CH₂).

MS (FAB+) m/z (244, MH⁺).

### EXAMPLE 13

### 6-Amino-N-trifluoroacetyl-1-aminoindan (12(s6)) 13.1: 6-Nitro-N-trifluoroacetyl-1-aminoindan (11(s6))

### 13.1.1 From 6-nitro-1-aminoindan

b 6-Nitro-1-aminoindan (9(s6), 17.92 g, 0.1 mol; prepared either by nitration of 1-aminoindan or by deacetyalation of 6-nitro-1-acetylaminoindan) was dissolved in acetonitrile (60 ml) and added dropwise to a solution of trifluoroacetic anhydride (23.10 g, 15.5 ml, 0.11 mol) in acetonitrile (60 ml) at 0-5° C. over a period of 0.5 h. Potassium hydroxyde (7.86 g, 0.14 mol) in water (100 ml) was added dropwise to the reaction mixture within ten minutes. The mixture was stirred for 3 h, and diluted with water (200 ml). The solid was collected by filtration, washed with water and dried. The crude product (23.9 g) was dissolved in methanol (310 ml) at rt, treated with charcoal, and water (155 ml) was added to the filtrate. The solid was collected by filtration and washed with 50% methanol. A second crop was obtained by concentrating the mother liquour. The two crops were combined and dried to give 21.5 g (78.4%), mp: 162-164° C.

¹H-NMR (DMSO-d₆) δ : 9.9 (br s, 1H, NH), 8.20 (d, 1H, Ar), 8.0 (s, 1H, Ar), 7.55 (s, 1H, Ar), 5.41 (br s,1H, C1-H), 3.15 (m, 1H, C3-H), 2.96 (m, 1H, C3-H′), 2.55 (m, 1H, C2-H), 2.06 (m, 1H, C2-H′) ppm.

### 13.1.2 From N-trifluoroacetyl-1-aminoindan

Conc. sulfuric acid (123.9 ml) was added to a cooled mixture of 65% nitric acid (7.7 ml, 0.11 mol) and water (19.4 ml), and the mixture added dropwise to a stirred and cooled at (2-8° C.) suspension of 1-trifluoroacetylaminoindan (10, 22.92 g, 0.1 mol) in nitromethane (170 ml) The mixture was stirred for 1.5 h and poured onto a mixture of ice (500 g) and water (1300 ml). The resulting mixture was stirred for 1 h and filtered. The collected solid was washed with water and dried. The crude product (22 g) was crystallized twice from methanol-water to give 5.5 g (20%). This product was identical to that obtained in Ex. 13.1.1.

¹H-NMR (DMSO-d₆) δ : 9.78 (d, 1H, NH), 6.95 (d, 1H, Ar), 6.52 (d, 1H, Ar), 6.45 (s, 1H, Ar), 5.28 (q,1H, C1-H), 5.0 (br s, 2H, NH₂), 2.80 (m, 1H, C3-H), 2.71 (m, 1H, C3-H′), 2.38 (m, 1H, C2-H), 1.92 (m, 1H, C2-H′) ppm.

### 13.2 6-Amino-N-trifluoroacetyl-1-aminoindan (12(s6))

A mixture of 6-nitro-1-trifluoroacetylaminoindan (11(s6), 27.42 g, 0.1 mol), iron dust (55.85 g, 1 gram atom), ethanol (170 ml), water (25 ml) and conc HCl (1 ml) was refluxed for 3 h and filtered while hot. The collected solid was boiled in ethanol (170 ml) and the mixture filtered. The combined filtrates (about 340 ml) were treated with charcoal, filtered and concentrated to a small volume under reduced pressure. The crystals were collected by filtration, washed with cold ethanol and dried to give 22.7 g (93%), mp: 133-134° C. Elemental analysis: calc. C, 54.10; H, 4.54; N, 11.47. Found: C, 54.11; H, 4.61; N, 11.48.

### EXAMPLE 14

### N₅-prop-2-ynyl-6,7-dihydro-5H-1-thia-3-aza-s-indacene-2,5-diamine 2HCl (A-1)

### 14.1 N₅-trifluoroacetyl-6,7-dihydro-5H-1-thia-3-aza-s-indacene-2,5-diamine (14)

6-Amino-N-trifluoroacetyl aminoindan (12(s6)), 2 g, 8.18 mmol) and NH₄SCN (1.44 g, 18.9 mmol) were dissolved in AcOH (25 ml), and Br₂ (1.44 g in 9 ml AcOH, 9 mmol) was added within 1 h at rt, followed by 15 ml AcOH. The viscous mixture was stirred first at 50-60° C. for 2-3 h, then at rt for 24 h. Water (200 ml) was added and the acidic mixture was neutralized with Na₂CO₃ followed by NaOH to pH 14. The solid was collected by filtration, washed with water and dried to give a yellow solid (1.6 g, 65%).

### 14.2 6,7-dihydro-5H-1-thia-3-aza-s-indacene-2,5-diamine (15)

The solid obtained in Ex. 14.1 (1.6 g, 5.3 mmol) and K₂CO₃ (1 g, 7.23 mmol) were suspended in a 2:1 MeOH:water mixture (21 ml) and stirred at 70-80° C. for 3h, and then at rt overnight. The solvents were evaporated and the residue partially purified by chromatography to yield a ca 5:1 mixture of the two regioisomers (425 mg).

¹H NMR (mixture of isomers, DMSOd₆): δ 7.41 (s, 1H, Ar major isomer), 7.32 (s, 1H, Ar major isomer), 7.29 (br s, NH₂ major isomer), 7.25 (br s, NH₂ minor isomer), 7.14 (d, 1H, J=8 Hz, Ar minor isomer), 4.26 (t, 1H, J=8 Hz, CHNH₂, minor isomer), 4.17 (t, 1H, J=7 Hz, CHNH₂, major isomer), 2.83 (ddd, 2H, J=15, 8, 3 Hz, CHCH₂CH₂ two isomers), 2.71 (m, 2 H, CHCH₂CH₂ two isomers), 2.28-2.44 (m, 2 H, CHCH₂CH₂ two isomers), 1.60 (dq, 2H, J=13, 8 Hz, CHCH₂CH₂ two isomers).

¹H NMR (minor isomer) δ: 7.23 (br s, 2H, NH₂), 7.14 (d, 1H, J=8 Hz, Ar), 7.02 (d, 1H, J=8 Hz, Ar), 4.25 (t, 1H, J=8 Hz, CHNH₂), 2.87 (ddd, 1H, J=15, 9, 4 Hz, CHCH₂CH₂), 2.74 (dt, 1 H, J=15, 9 Hz, CHCH₂CH₂), 2.44 (dddd, 1H, J=15, 9, 8, 4 Hz, CHCH₂CH₂), 1.67 (dq, 1H, J=15, 9 Hz, CHCH₂CH₂).

¹³C (major isomer, DMSOd₆) δ: 165.69 (NCNH₂), 151.66, 146.56, 135.62, 129.33 (Ar), 116.05, 113.04 (Ar CH), 56.67 (CHNH₂), 37.42 (CHCH₂CH₂), 29.26 (CHCH₂CH₂).

¹³C (minor isomer, DMSOd₆) δ: 166.66 (NCNH₂), 152.67, 140.03, 134.91, 125.89 (Ar), 121.33, 116.25 (Ar CH), 57.25 (CHNH₂), 38.51 (CHCH₂CH₂), 29.77 (CHCH₂CH₂).

TOF MS (206, MH⁺), (189, MH⁺—NH₃).

### 14.3 N₅-prop-2-ynyl-6,7-dihydro-5H-1-thia-3-aza-s-indacene-2,5-diamine (free base of A-1)

A mixture of the isomeric mixture obtained in Ex. 14.2 (350 mg, 1.7 mmol), propargyl bromide (127 μl, 1.7 mmol) and K₂CO₃ (0.276 g, 2 mmol) in acetonitrile (10 ml) was heated at 85° C. for 3 h. The solvent was evaporated, and the crude product was purified by chromatography to give the title product as an oil (0.125 g, 30%).

¹H NMR (DMSOd₆) δ: 7.44 (s, 1H, Ar), 7.34 (br s, 2 H, NH₂), 7.32 (s, 1H, Ar), 4.25 (t, 1H, J=6 Hz, CHNH₂,), 3.39 (ABq of d of d, 2H, Jⱼₑₘ=15 , J_{vic}=3 Hz, NCH₂), 3.07 (t, 1H, J=3 Hz, CH₂CCH), 2.90 (ddd, 1H, J=15, 9, 4 Hz, CHCH₂CH₂), 2.71 (dt, 1H, J 15, 9 Hz, CHCH₂CH₂), 2.29 (dddd, 1H, J=15, 9, 8, 4 Hz, CHCH₂CH₂), 1.67 (dq, 1H, J=15, 9 Hz, CHCH₂CH₂).

¹³C (DMSOd₆) δ: 165.89 (NCNH₂), 151.43, 143.08, 136.38, 129.95 (Ar), 116.37, 113.56 (Ar CH), 83.39, 73.49 (NCH₂CCH), 61.10 (CHNH₂), 35.46 (NCH₂), 33.41 (CHCH₂CH₂), 29.60 (CHCH₂CH₂).

MS (DCI) (CH₄) m/z (243, (M-H)⁺)

### EXAMPLE 15

### 2-Amino-6,7-dihydro-1-thia-3-aza-as-indacen-8-one (21)

Ammonium thiocyanate (2.0 g, 26.3 mmol) was added to a solution of 6-amino indanone (13(s6), 1.66 g, 11.3 mmol) in glacial HOAc (20 ml). The mixture was stirred until a clear solution was obtained, heated to 40° C. under N₂, at which point a solution of bromine (2.0 g, 12.5 mmol) was added dropwise, while maintaining the temperature at 40-45° C. The resulting suspension was further stirred at 40° C. for 2 h, cooled and water (150 ml) was added, followed by Na₂CO₃ to a pH of 9-10. The mixture was stirred at rt for 2 h and filtered. The collected solid was washed with water and hexane, dried and column-chromatographed (CH₂Cl₂:MeOH 90:10) to give 463 mg (20 %) of a brown solid.

¹H-NMR (DMSO-d₆) δ: 7.63 (d, 1H, Ar), 7.40 (d, 1H, Ar), 7.60 (br s, 2H, NH₂), 3.15 (m, 2H, C3-H), 2.70 (m, 2H, C2-H) ppm.

### EXAMPLE 16

### 2-Amino-6,7-dihydro-1-thia-3-aza-as-indacen-8-one oxime (22)

A mixture of 2-Amino-6,7-dihydro-1-thia-3-aza-as-indacen-8-one (21, 0.6 g, 2.94 mmol), hydroxylamine HCl (0.66 g, 9.50 mmol) and sodium acetate (0.84 g, 10.24 mmol) in 2:1 EtOH:water mixture (18 ml) was refluxed under N₂ for 24 h. The mixture was cooled to rt, water (100 ml) was added, stirred for 15 min and filtered. The collected solid was washed with water and hexane and dried to give 480 mg (75%) of a tan solid.

¹H-NMR (DMSO-d₆) δ: 11.10 (s, 1H, NOH), 7.45 (br s, 2H, NH₂), 7.35 (d, 1H, Ar), 7.21 (d, 1H, Ar), 3.05 (m, 2H, C3-H), 2.86 (m, 2H, C2-H) ppm.

### EXAMPLE 17

### N₁-Methyl-N₁-prop-2-ynyl-indan-1, 6-diamine (6-amino-N-methyl-propargylaminoindan, 25 (s6))

### 17.1 Methyl-(6-nitro-indan-1-yl)prop-2-ynyl-amine (6-nitro-N-methyl-propargylaminoindan (24 (s6))

A mixture of 6-nitro-1-propargylaminoindan (4.32 g, 20 mmol), paraformaldeyde (2.69 g), NaCNBH₃ (1.47 g, 23.4 mmol) in absolute MeOH (80 ml) was refluxed under nitrogen for 4 h. Silica (10 g) was added to the clear solution, and the mixture evaporated to dryness under reduced pressure. The silica-impregnated crude product was purified by flash column chromatography (75:25 EtOAc:hexane) to give 4.42 g (96%) of yellow oil.

### 17.2 N₁-Methyl-N₁-prop-2-ynyl-indan-1, 6-diamine (6-amino-N-methyl-propargylaminoindan, 25 (s6))

6-Nitro-N-methyl-propargylaminoindan (2.18 g, 9.48 mmol) was dissolved in anhydrous EtOH (60 ml), and stannous chloride dihydrate (10.8 g, 47.9 mmol) was added. The clear solution was stirred for 20 h under nitrogen at ambient temperature, and triethylamine (12 ml) was added. The resulting suspension was diluted with EtOH (100 ml) and filtered. The filtrate was evaporated to dryness, and EtOAc (150 ml) was added to the residue. The mixture was stirred for 10 min and filtered. The filtrate was washed with water, and the organic layer was separated, dried and evaporated to dryness to give a viscous orange oil (1.5 g). This crude product was further purified by converting it to the dihydrochloride salt (HCl/Et₂O). The salt was converted to the free base (aqueous ammonia), followed by flash column chromatography of the latter (90:10 CH₂Cl₂/MeOH) to give the title compound as a viscous tan oil (1.14 g, 60%.

¹H NMR (DMSO-d₆) δ : 1.94 (m, 2H, C2-H), 2.56 (m, 1H, C3-H), 2.68 (m, 1H, C3-H′), 3.11 (t, J=1, 2.5Hz, CH₂CCH), 3.26 (d, J=2.5 Hz, 2H, CH₂CCH), 4.23 (t, J=7 Hz, 1H, C1-H), 4.84 (brs, 2H, NH₂), 6.42 (dd, J=8, 2 Hz, 1H, C5-H), 6.51 (d, J=2 Hz, 1H, C7-H), 6.85 (d, J=8 Hz, 1H, C4-H).

MS: 201 (MH⁺, 100), 162 (MH⁺—C₃H₃, 70), 133 (MH⁺—MeNC₃H₃, 95).

### EXAMPLE 18

### N₁-prop-2-ynyl-indan-1,6-diamine (6-amino-N-propargylaminoindan, 8(s6))

To a solution of 6-nitro-propargylaminoindan mesylate (0.5 g, 1.6 mmol) in anhydrous EtOH (15 mL) was added stannous chloride dihydrate (1.8 g, 8 mmol), and the mixture was stirred at rt under nitrogen for 4.5 h to give a yellow suspension. It was diluted with EtOH, the pH was adjusted to 10 by Et₃N, and filtered. The filtrate was evaporated to dryness, and the residue treated with EtOAc. The suspension was filtered, and the filtrate was washed with water; the phases were separated and the organic phase was dried (NaSO₄), filtered and evaporated to dryness to give the title compound as a yellow oil (25%).

¹H-NMR (DMSO-d₆) δ 1.68 (m, 1H, C2-H), 2.20 (m, 1H, C2-H′), 2.54 (m, 1H, C3-H), 2.70 (m, 1H, C3-H′), 3.06 (t, J=1, 1H, CH₂CCH), 3.35 (m, 2H, CH₂CCH), 4.10 (t, J=6 Hz, 1H, C1-H), 4.81 (br s, 2H, NH₂), 6.40 (dd, J=8 Hz, 1H, C5-H), 6.55 (d, J=2 Hz, 1H, C7-H), 6.85 (d, J=8 Hz, 1H, C4-H).

MS: 186 (M⁺, 36), 147 (15), 131 (100).

### EXAMPLE 19

### Effect of Compounds A-1 and B-1 on MPP+ Treated PC-12 Cells

Pheochromocytoma PC-12 cells (at a density of 200,000 cells/well) were cultured for 10 days with 50 ng/ml NGF on 6-well culture dishes coated with 200 pg/ml rat tail type I collagen (BD Biosciences, Bedford, Mass., USA). At the day of the experiment the morphological differentiation of the cells was very advanced (typical network formation). In order to initiate the neurotoxic insult, cells were treated with 1000 μM of 1-methyl-4-phenylpyrdinium (MPP+) iodide salt from RBI chemicals (Natick, Mass., USA) for 48 hours in the absence or presence of tested compounds, added to the culture 30 min. prior to MPP+ administration. MPP+ has been shown to inhibit mitochondrial electron transport (complex I) in neurons and to induce a syndrome resembling Parkinson's disease in mice and monkeys. At the cellular level, neuronal cell death is induced by several mechanisms including pathological concentrations of intracellular calcium and free oxygen radicals. Therefore the positive controls in this experiment were nimodipine at the concentration of 10 μM (RBI chemicals, Natick, Mass., USA) (a potent L-type calcium channel blocker) and 4-hydroxy-2,2,6,6-tetramethylpiperidyne-1-oxyl (tempol, a potent antioxidant) at a concentration of 500 μM, (Sigma, St Louis, Mo., USA). At the end of the experiment cell death was measured by assessing Lactate dehydrogenase (LDH) activity in the medium. High medium LDH indicated increased neuronal death that promoted leakage of this cytoplasmic enzyme into the medium.

Measuring lactate dehydrogenase activity in the medium was performed using a Sigma Diagnostics LD-L reagent. LDH activity was spectrophotometrically monitored at 340 nm by following the rate of conversion of oxidized nicotinamide adenine dinucleotide (NAD⁺) to the reduced form of (NADH). Total LDH of each culture (extracellular+intracellular) was obtained by measuring LDH in the medium after freezing and thawing of the cultures. Basal LDH release was measured in untreated cultures (no MPP+). The neurotoxic effect was calculated according to the formula: (LDHs−LDHb)/LDHt×100. (s=sample; b=basal; t=total). Each compound was tested in sixplicate.

Results are summarized in the table below. To calculate the percentage of neuroprotection for each set of sixplicate experiments, the average neurotoxicity was calculated and the following calculation was undertaken: 100−([neurotoxicity test compound/neurotoxicity MPP⁺]×100).

TABLE 1

| Compound | Dose | | % neuroprotection |
|---|---|---|---|
| A-1 | 1 | μM | 50 |
| | 10 | μM | 60 |
| B-1 | 1 | μM | 60-87 |
| | 10 | μM | 58-81 |
| Tempol | 1000 | μM | 100 |

### EXAMPLE 20

### Activity of Compounds A-1 and B-1 in the Experimental Allergic Encephalomyelitis (“EAE”) Model of MS

EAE was induced by injecting the encephalitogenic agent consisting of MSCH and commercial CFA containing Mycobacterium tuberculosis H37Ra to the foot-pads of the animals and pertussis toxin intravenously. The mice were allocated to the following treatment groups (10 mice/group):

TABLE 2

| | | Oral | Administration |
|---|---|---|---|
| Group | Group identification | dose/day | Route |
| 1 | Control PBS | 0 | — |
| 5 | A-1 (10 mg/kg twice a day) | 20 mg/kg | oral |
| 4 | A-1 (5 mg/kg twice a day) | 10 mg/kg | oral |
| 7 | B-1 (10 mg/kg twice a day) | 20 mg/kg | oral |
| 6 | B-1 (5 mg/kg twice a day) | 10 mg/kg | oral |

Compounds A-1 and B-1 were administered in 0.5% methyl-cellulose.

EAE symptoms were evaluated on the following scale:

0 = no clinical symptoms
1 = loss of tail tonicity
2 = partial hind limb paralysis
3 = complete hind limb paralysis
4 = paralysis of four limbs
5 = death.

The results are summarized in FIGS. 1(a)-(d) and in Table 3.

TABLE 3

| | | incidence | | GMS | | GMS | | Mean |
|---|---|---|---|---|---|---|---|---|
| Group | | | % | | % | | % | duration |
| No. | treatment | Value | inhibition | Value | inhibition | Value | inhibition | (days) |
| 1 | Control | 4/10 | | 1.0 ± 1.4 | | 0.6 ± 0.9 | | 6.1 ± 8.7 |
| | (methyl | | | | | | | |
| | cellulose) | | | | | | | |
| 4 | A-1 | 2/10 | 50.0% | 0.6 ± 1.3 | 40.0% | 0.4 ± 0.8 | 33.3% | 3.0 ± 6.3 |
| | 2 × 5 mg/kg | | | | | | | |
| 5 | A-1 | 1/10 | 75.0% | 0.3 ± 0.9 | 70.0% | 0.2 ± 0.6 | 66.7% | 1.8 ± 5.7 |
| | 2 × 10 mg/kg | | | | | | | |
| 6 | B-1 | 1/10 | 75.0% | 0.1 ± 0.3 | 90.0% | 0.005 ± 0.02 | 99.2% | 0.1 ± 0.3 |
| | 2 × 5 mg/kg | | | | | | | |
| 7 | B-1 | 2/10 | 50.0% | 0.4 ± 1.0 | 60.0% | 0.3 ± 0.8 | 50.0% | 3.3 ± 7.0 |
| | 2 × 10 mg/kg | | | | | | | |

Discussion

MPP+ has been shown to inhibit mitochondrial electron transport (complex I) in neurons and to induce a syndrome resembling Parkinson disease in mice and monkeys. As illustrated in the preceding examples, the compounds of the invention are effective at preventing neuronal cell death of MPP+ treated PC-12 cells and in mice treated with MPP+. Consequently, the results indicate that the compounds of the invention would be effective in treating Parkinson's disease in humans.

In addition, MMPs are increasingly being implicated in the pathogenesis of multiple sclerosis (reviewed in Yong et al., Trends Neurosci., 1998, 21:75-80; Kieseier et al., Neurol., 1999, 53:20-25). At least 4 different mechanisms are thought to contribute to the role that MMPs may play in multiple sclerosis. First, MMPs are produced by leukocytes to degrade the basement membrane surrounding blood vessels and hence they disrupt the integrity of the blood-brain barrier. Second, MMPs are utilized by leukocytes to remodel the brain ECM in order to gain entry into the CNS parenchyma. Recent evidence suggests that the expression of matrix metalloproteinases (MMPs) by leukocytes is required for T lymphocytes to enter the CNS parenchyma (Leppert et al. Ann. Neurol., 1996, 40:846-852; Stuve et al. Ann. Neurol., 1996, 40:853-863; Xia et al. J. Immunol., 1996, 156:160-167;). Third, proteolytic processing by MMPs results in the formation of the oligodendrocyte-toxic cytokine, TNF-α, from its pro-form. Other molecules believed to be processed by MMPs include FasL, IL-6 receptor, and ICAM. Fourth, the intracerebral injection of purified MMPs results in the direct degradation of the myelin sheath.

Furthermore, experimental allergic encephalomyelitis (EAE) is an inflammatory demyelinating disease of the central nervous system (CNS) that serves as a model for the human demyelinating disease, multiple sclerosis (MS). As illustrated in Example 18 above, the compounds of the invention are effective in treating EAE in mice. Thus, the results indicate that the compounds of the invention would be effective in treating multiple sclerosis in humans.

1. A compound having the structure: 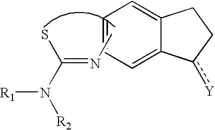
wherein
Y is O, NR₃R₄ or NOR₆;
R₃ is H, alkyl, aralkyl, alkynyl, trifluoroacetyl, t-butoxycarbonyl or an acyl group;
R₄ is H, alkyl, aralkyl, or alkynyl;
R₆ is H or C₁-C₄ alkyl;
R₁ and R₂ are each independently H, alkyl, aralkyl, or alkynyl;
the curved line drawn from S to the center of the phenyl ring and the straight line drawn from N to the center of the ring indicate that S and N are part of a 5 membered ring which shares two carbons with the phenyl ring; and
the dashed line drawn from the carbon atom on the cyclopentyl ring to Y represents a bond when Y is O or NOR₆ and is absent when Y is NR₃R₄,
or an enantiomer, or a tautomer, or a pharmaceutically acceptable salt thereof.

2. The compound of claim 1, having the structure: 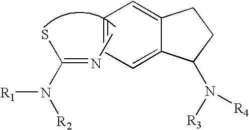
wherein,
R₁, R₂, and R₄ are each independently H, alkyl, aralkyl, or alkynyl;
R₃ is H, alkyl, aralkyl, alkynyl, trifluoroacetyl, t-butoxycarbonyl or an acyl group; and
the curved line drawn from S to the center of the phenyl ring and the straight line drawn from N to the center of the ring indicate that S and N are part of a 5 membered ring which shares two carbons with the phenyl ring,
or an enantiomer, or a tautomer, or a pharmaceutically acceptable salt thereof.

3-22. (canceled)

23. The compound of claim 1, having the structure: 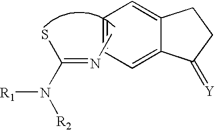
wherein Y is O or NOR₆, and
R₁, R₂ and R₆ are as defined above.

24. The compound of claim 23, having the structure:

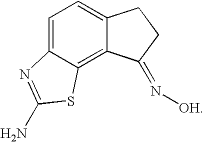

25. The compound of claim 23, having the structure:

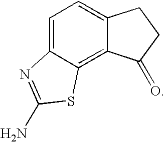

26. A compound having the structure: 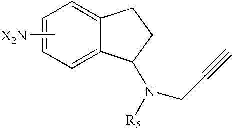
wherein
X is H or O; and
R₅ is H, alkyl, trifluoroacetyl, t-butoxycarbonyl or an acyl group,
or an enantiomer, or a tautomer, or a pharmaceutically acceptable salt thereof.

27-36. (canceled)

37. A compound having the structure: 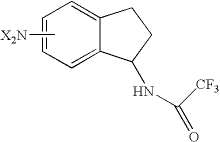
wherein X is H or O.

38. The compound of claim 37, wherein X is O.

39. A process for manufacturing the compound of claim 38 comprising reacting 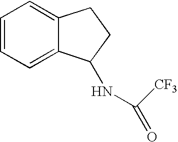
with nitromethane in the presence of sulfuric acid and nitric acid to produce the compound.

40. A process for manufacturing the compound of claim 38 comprising reacting 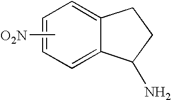
with trifluoroacetic anhydride and potassium hydroxide in the presence of acetonitrile to produce the compound.

41. The compound of claim 37, wherein X is H.

42. A process for manufacturing the compound of claim 41 comprising reacting 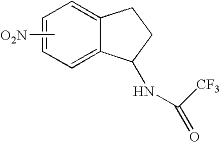
with iron dust and hydrochloric acid in the presence of ethanol to produce the compound.

43. A method of treating a subject suffering from Parkinson's disease or multiple sclerosis, comprising administering to the subject a therapeutically effective amount of the compound of claim 2 so as to thereby treat the subject.

44-46. (canceled)

47. A method for treating a subject suffering from depression comprising administering to the subject a therapeutically effective amount of the compound of claim 2, so as to thereby treat the subject.

48. A pharmaceutical composition comprising a compound of claim 2 and a pharmaceutically acceptable carrier.

49-52. (canceled)

53. A process of manufacturing the compound of claim 26, wherein X is H or O and R₅ is H comprising
reacting 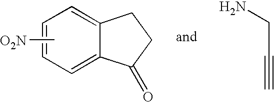
in the presence of Na(OAc)₃BH and DCE to produce

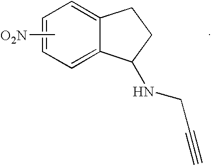

54. A process of manufacturing the compound of claim 26, wherein X is H or O and R₅ is H or alkyl, comprising reacting 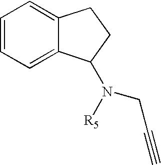
with a nitrating agent in the presence of acid to produce

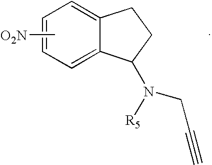

55-65. (canceled)

66. A process of manufacturing the compound of claim 2, comprising the steps of:
(a) reacting a compound having the structure: 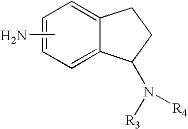
wherein
R₃ is trifluoroacetyl or t-butoxycarbonyl; and
R₄ is H, alkyl,aralkyl, or alkynyl;
with cyclizing agents in the presence of solvent; and
(b) removing the trifluoroacetyl or t-butoxycarbonyl group by reacting the product of step (a) with a suitable reagent to produce the compound.

67-70. (canceled)

71. A process of manufacturing the compound of claim 25 comprising reacting a compound having the structure 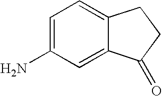
with cyclization agents in the presence of solvent to produce the compound.

72. A process of manufacturing the compound of claim 24, comprising refluxing the compound having the structure 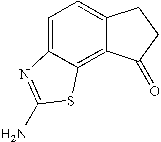
with NH₂OH.HCl, NaOAc, EtOH, and water to produce the compound.

73-92. (canceled)
